(19) United States
(12) Patent Application Publication
(10) Pub. No.: US 2010/0248269 A1
(43) Pub. Date: Sep. 30, 2010

(54) DETECTION OF FIBRIN AND FIBRINOGEN DEGRADATION PRODUCTS AND ASSOCIATED METHODS OF PRODUCTION AND USE FOR THE DETECTION AND MONITORING OF CANCER

(75) Inventor: Andrea Small-Howard, Norwalk, CA (US)

Correspondence Address: K&L Gates LLP, 1900 MAIN STREET, SUITE 600, IRVINE, CA, 92614-7319, US

(73) Assignee: New York Blood Center, New York, NY (US)

(21) Appl. No.: 12/414,518

(22) Filed: Mar. 30, 2009

(51) Int. Cl. G01N 33/00; G01N 33/53

(52) U.S. Cl. 435/7.92; 435/7.1

(57) ABSTRACT

Disclosed herein are methods, systems and kits for the detection of, or monitoring the progression of, cancer by simultaneously detecting the presence of six fibrin and fibrinogen degradation products (FDP) in a biological sample in a single assay system.

### FIELD OF THE INVENTION

The present application relates generally to methods of producing and to the production of antibody populations against fibrinogen and fibrin degradation products (FDP), to the antibody populations themselves and to related methods of use, to the detection of cancers and for monitoring the progress of cancer treatment by immunochemically measuring the quantity of FDP in serum.

### BACKGROUND OF THE INVENTION

Despite recent advances in the understanding of cancer, current techniques for the screening and identification of cancer leave room for improvement. Methods known in the art for screening cancer attempt to detect cancer-related antigens by using antibodies. Antigens are macromolecules, such as proteins, nucleic acids or polysaccharides, which are capable of eliciting an immune response in the body. The immune systems of mammals and other animals have the ability to detect foreign agents, such as the antigens associated with cancer, and to respond to these antigens by producing antibodies which specifically target and react with those cancer-associated antigens. Thus, there is a strong correlation between the detection of these cancer-associated antigens or the circulating antibodies that target these antigens in a mammal's circulating blood and the existence of cancer in that individual. As a result, tests that detect the existence of such antigens or antibodies are useful in the screening and diagnosis of cancer.

One promising cancer-associated antigen target is a pool comprised of both fibrin and fibrinogen degradation products (FDP). While the production of both fibrin and fibrinogen degradation products is restricted in healthy individuals, FDP are over produced in cancer patients when proteolytic enzymes, such as plasmin and thrombin, are released by cancer cells. In addition, FDP are produced as a by-product of other cancer-related processes, such as angiogenesis and metastasis. Because FDP leak from tumors into surrounding fluids, elevated FDP levels can be measured in the urine of subjects with bladder cancer, in the plasma of lung cancer subjects, and in the plasma or serum of other cancer patients. The utility of FDP measurements in cancer diagnostics has been suspected for years; however refined assays had not been developed that were able to quantitatively measure FDP with the sensitivity required. Current assays for FDP are usually restricted to measuring one specific FDP component, such as D-dimer, as a representative of this group.

Cancer elevates fibrin and fibrinogen degradation product (FDP) levels both through the tissue factor (TF) and urokinase-type plasminogen activator (uPA) regulated pathways generating both fibrinogen degradation products and fibrin degradation products (FIG. 1). Cleavage of fibrinogen by plasmin produces fragments D and E, as the primary end-products. Thrombin converts fibrinogen to fibrin in response to signals from the coagulation cascade. Cleavage of fibrin by plasmin produces D-dimer as a primary end-product. The composition of FDP produced through cancer-induced plasmin cleavage is influenced by the relative amounts of the two substrates; fibrin and fibrinogen.

### SUMMARY OF THE INVENTION

Disclosed herein are methods for the detection of cancer comprising the simultaneous detection of a defined mixture of fibrin and fibrinogen degradation products (FDP) containing three essential fragment types (fragment D, fragment E, and D-dimer) and optionally an additional three fragment types (fragment Y and two distinguishable forms of initial plasmin digest product—IPDP) for a total of six FDP components. The method comprises the simultaneous detection of the three essential FDP antigens in a biological sample with a specific polyclonal antibody pool in which the polyclonal antibodies are specific for the three FDP fragments.

In one embodiment of the present disclosure, a method is provided for detecting cancer in a subject, the method comprising the steps of: obtaining a biological sample from the subject; reacting the biological sample with an antibody preparation which binds at least three antigens associated with fibrin and fibrinogen degradation products (FDP) to form antibody-FDP complexes wherein the three FDP-associated antigens are fragment D, fragment E and D-dimer; detecting the antibody-FDP complexes; and diagnosing cancer in the subject. In another embodiment, the antibody preparation additionally optionally binds to at least one of fragment Y and initial plasmin digest products (IPDP). In another embodiment, the antibody preparation is a polyclonal antibody preparation.

In another embodiment, the method comprises an enzyme-linked immunosorbent assay.

In another embodiment, the diagnosing step further comprises at least one additional diagnostic test.

In one embodiment of the present disclosure, a method is providing for monitoring cancer in a patient comprising the steps of: (a) obtaining a first biological sample from the subject, the first sample collected at a first sampling time point; (b) obtaining a second biological sample from the subject, the second sample collected after the first sampling time point at a second sampling time point; (c) reacting the biological samples with an antibody preparation which binds at least three antigens associated with FDP to form antibody-FDP complexes wherein the three FDP-associated antigens are fragment D, fragment E and D-dimer; (d) detecting the antibody-FDP complexes; (e) determining the ratio of the level of FDP in the second biological sample to the level of FDP in the first biological sample; (f) determining that cancer has progressed; and optionally repeating steps (a)-(e) with additional biological samples taken at time points after the first and the second time points.

In another embodiment, the antibody preparation additionally optionally binds to at least one of fragment Y and initial plasmin digest products (IPDP). In another embodiment, the antibody preparation is a polyclonal antibody preparation.

In another embodiment, the method comprises an enzyme-linked immunosorbent assay.

In another embodiment, the cancer has progressed if the ratio is greater than or equal to 1.15. In yet another embodiment, the cancer has regressed or is stable if the ratio is less than 1.15.

In one embodiment of the present disclosure, a kit is provided for detecting cancer in a subject, the kit comprising an antibody preparation which binds to at least three antigens associated with FDP wherein the three FDP-associated antigens are fragment D, fragment E and D-dimer; a detection system; and instructions for measuring the FDP and correlating the presence of the FDP with cancer.

In another embodiment, the detection system comprises a detection antibody specific for at least three antigens associated with FDP wherein the three FDP-associated antigens are fragment D, fragment E and D-dimer. In another embodiment, the antibody preparation optionally additionally binds to at least one of fragment Y and initial plasmin digest products (IPDP).

In another embodiment, the antibody and detection system comprise an enzyme-linked immunosorbent assay.

### BRIEF DESCRIPTION OF THE DRAWINGS

FIG. 1 depicts a schematic representation of cancer-induced generation of fibrin and fibrinogen degradation products (FDP).

FIG. 2 depicts non-reducing (FIG. 2A) and reducing (FIG. 2B) SDS-PAGE gels of FDP immunogen lots (8 lots designated 03, 13, 15, 17, 19 21, 24 and 25). UD refers to undigested fibrinogen; D refers to fragment D; and E refers to fragment E.

FIG. 3 depicts a Western Blot analysis of anti-FDP antibody binding to human serum samples (FIG. 3A, exposed to film for approximately 5 sec and FIG. 3B, exposed to film for approximately 30 sec) from colorectal carcinoma (CRC) patients (n=5) and normal control serum (n=5). FIG. 3C depicts Coomassie Blue staining of the companion gel depicting the total amount of protein loaded per lane. The location of FDP subtypes are indicated on the gels.

FIG. 4 depicts immune precipitation (IP) of antigens from human serum (normal and CRC patients) using anti-FDP polyclonal antibody pool (+) and rabbit IgG (−) bound beads. Three samples were tested: FDP calibrator control (FDP), a normal human serum (N4) and a CRC serum (C15).

FIG. 5 depicts FDP antigens immune precipitated from the sera of five CRC and five normal patients using anti-FDP polyclonal antibody pool (FIG. 5A) or rabbit IgG (control) bound beads (FIG. 5B).

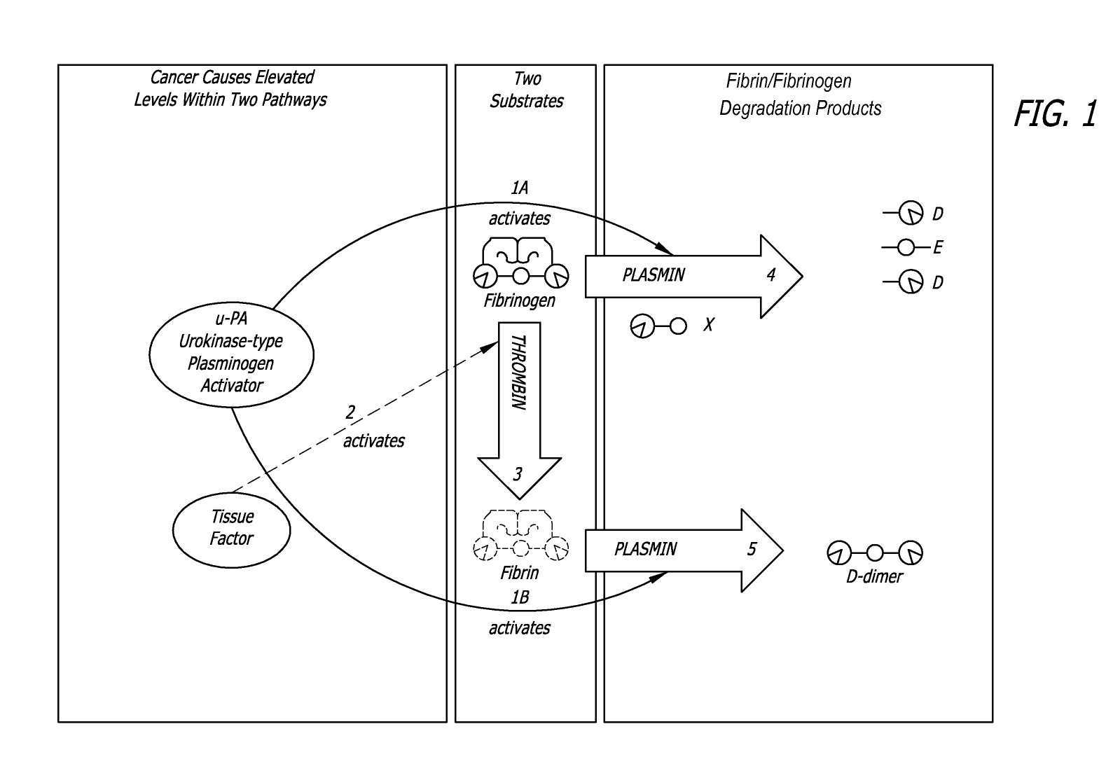

### DETAILED DESCRIPTION OF THE INVENTION

Detection of fibrin and fibrinogen degradation products (FDP) can be a valuable clinical diagnostic tool in a number of cancers. FDP levels are correlated with cancer occurrence, stage, progression and prognosis. Current assays for FDP are usually restricted to measuring one specific FDP component, such as D-dimer, as a representative of this group. In contrast, the present inventor has determined that a plurality of the fibrin and fibrinogen degradation products must be measured simultaneously in order to correlate the presence of, or a change in the levels of, FDP with the presence or progression of cancer, respectively. For the purposes of the present disclosure, the phrase “progression of cancer” includes the progression, regression or re-occurrence of cancer in a patient. The presently disclosed assay uses an antibody preparation which specifically recognizes at least three FDP antigens that are particularly useful in detecting, and monitoring, the progression of cancer. These three essential FDP are fragment D, fragment E and the D-dimer (also known as fragment X). Three additional FDP which are useful for detection and monitoring cancer are two initial plasmin digest products (IPDP), defined by sequencing, and fragment Y. In one embodiment of the instant method, the three essential FDP antigens are detected approximately simultaneously in the same assay. In other embodiments, four, five or six FDP antigens are detected simultaneously in the same assay.

In one embodiment, the disclosed assay measures fragment D, fragment E and D-dimer. In another embodiment, the disclosed assay measures fragment D, fragment E, D-dimer and one or both of the IPDP. In yet another embodiment, the disclosed assay measures fragment D, fragment E, D-dimer and fragment Y. In yet another embodiment, the disclosed assay measures fragment D, fragment E, D-dimer, fragment Y and one or both of the IPDP.

Immunoassays are well known in the art and the affinity-purified anti-FDP antibodies disclosed herein can be used in different assay methods including, but not limited to, enzyme-linked immunosorbent assays (ELISA), dot or slot blots, sepharose bead trapping analyte systems, or various rapid test formats, such as lateral-flow or flow-through systems.

One particular feature of the presently described method is that the capture and/or detection antibody is able to specifically bind to three, four, five or six FDP antigens. The antibody preparation is referred to herein as an antibody pool to reflect that the antibody preparation contains multiple antibody specificities. In one embodiment, the antibody preparation is a polyclonal antibody preparation raised against an immunogen preparation which contains three, four, five or six FDP antigen species. Polyclonal antibodies can be generated in a variety of species including, but not limited to rabbit, goat, horse, donkey, sheep, chicken or shark. In another embodiment, antibody preparations produced against each of the three, four, five or six FDP antigens can be prepared independently and pooled together to form the anti-FDP antibody pool.

Furthermore, in other embodiments, the antibody preparation can comprise a pool of monoclonal, monospecific polyclonal, chimeric or humanized antibodies or antibody fragments in which each antibody reacts with one or more of the six FDP antigens as long as the antibody pool can react with three, four, five or six FDP antigens in the same assay approximately simultaneously. The preparation of such antibodies is well known in the art and will not be described in detail in this disclosure.

The present disclosure also includes systems and kits for binding to FDP and detecting, or monitoring, the presence or progression of cancer in a subject. In one embodiment, the systems comprise antibody pools and detection reagents for binding and detecting three, four, five or six FDP in biological samples and correlating the presence of the three, four, five or six FDP with the presence or progression of cancer in the subject.

In one embodiment, the kits comprise antibody pools and detection reagents for binding and detecting three, four, five or six FDP in biological samples and for correlating the presence of the three, four, five or six FDP with the presence or progression of cancer in the subject. In another embodiment, a kit can contain one or more of the following in a package or container: (1) one or more antibody pools which bind three, four, five or six FDP to capture FDP from the biological sample; (2) detection antibody; (3) a solid support for immobilizing at least one of (a) the antibody pool or (b) the detection antibody; (4) detection reagents; (5) wash solutions; and (6) instructions for performing an assay using the kit components and biological samples. Embodiments in which two or more of components (1)-(6) are found in the same container can also be used.

When a kit is supplied, the different reagents can be packaged in separate containers and admixed or attached to the solid support immediately before use. Such packaging of the components separately can permit long-term storage without losing the active components' functions.

The compositions included in particular kits can be supplied in containers of any sort such that the life of the different components are preserved and are not adsorbed or altered by the materials of the container.

As stated earlier, kits can also be supplied with instructional materials. Instructions may be printed on paper or other substrate, and/or may be supplied as an electronic-readable medium, such as a floppy disc, CD-ROM, DVD-ROM, Zip disc, videotape, audiotape, flash memory device, etc. Detailed instructions may not be physically associated with the kit; instead, a user may be directed to an internet web site specified by the manufacturer or distributor of the kit, or supplied as electronic mail.

Additionally the claimed methods, systems and kits can be used to detect cancer-associated FDP in biological samples from a variety of sources including, but not limited to, serum, whole blood, plasma, saliva, sputum, urine, peritoneal fluid, tumor tissue, cerebrospinal fluid, vaginal or rectal secretions, and tears.

When using the assay method as a screen for cancer, each laboratory must establish its own normal and abnormal ranges, which are based on local population studies. Example 11 provides a typical distribution of normal and cancer patient samples. Levels of FDP in biological samples from patients suspected of having cancer are correlated with the presence of cancer by identifying those samples that exceed the normal range by a predetermined number.

When using the assay method for monitoring a patient with a confirmed cancer diagnosis, a baseline reading should precede the evaluation of their FDP levels. The FDP value of the initial serum draw serves as the baseline reading. The value of successive serum draws are evaluated by constructing the following ratio (R); where a=the initial FDP value and b=the current FDP value.

R=b/a

When the ratio of the current FDP value relative to the baseline FDP value is greater than 1.15, the patient is likely to have disease progression, however, results of FDP antigen testing should be used in conjunction with other clinical modalities that are standard of care for monitoring disease progression in these cancer patients.

### EXAMPLES

Commercially available reagents referred to in the examples were used according to manufacturer's instructions unless otherwise indicated.

### Example 1

### Preparation of the FDP Immunogen

The FDP immunogen was prepared by purchasing a purified human fibrinogen product that contained both fibrin and fibrinogen; which is referred to as fibrin/ogen for the purposes of this procedure. The fibrin/ogen was then reacted with plasmin to form fibrin and fibrinogen degradation products (FDP) by the Haverkate and Timan procedure (Thromb. Res. 10:803-812, 1977). The plasmin degradation of the fibrin/ogen was controlled by running the reaction for a specific period of time and then stopping the reaction with a protease inhibitor cocktail.

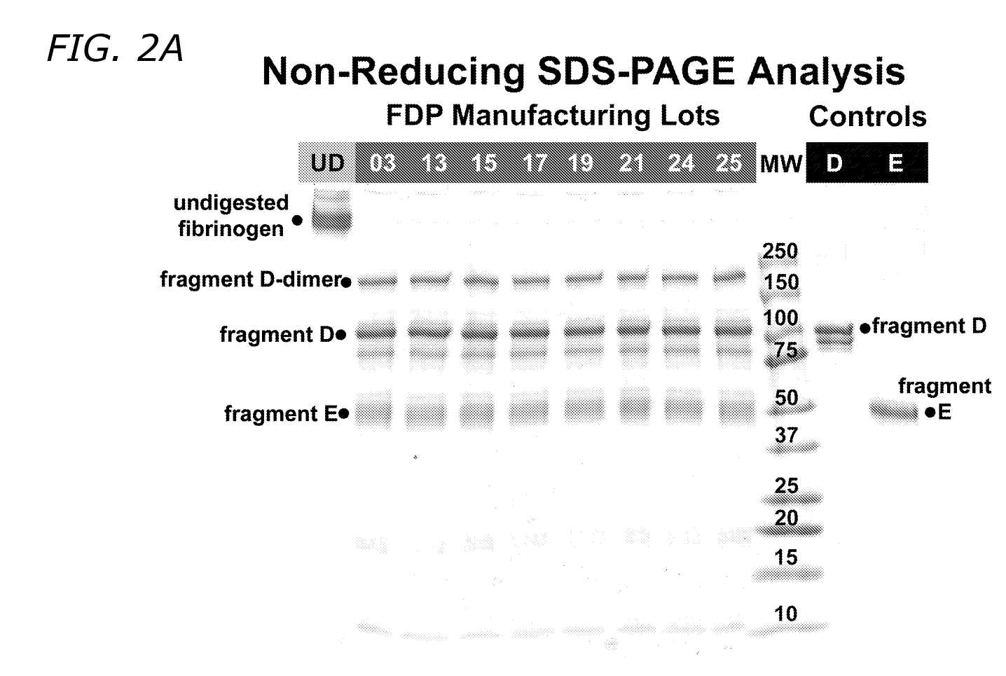

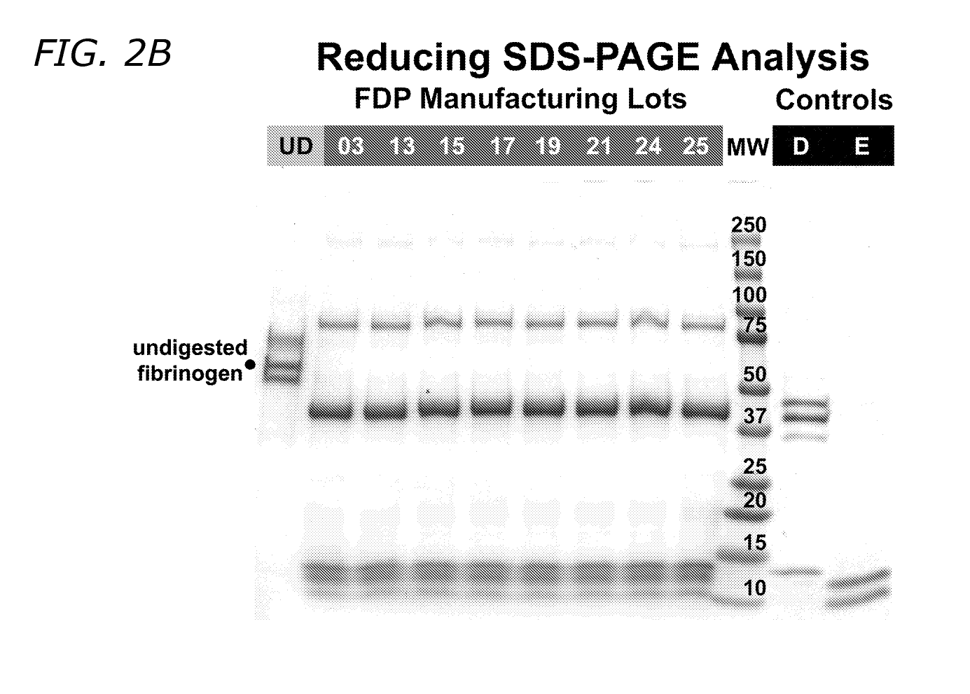

Specifically, the following procedure was used: Using a 50 ml conical tube, 20 ml of MOPS buffer (5 mM 3-(N-morpholino) propane-sulfonic acid, pH 7.4 containing 0.1 M sodium chloride and 20 mM calcium chloride) was incubated in 37° C. incubator until the solution reached 37° C. temperature (approx. 20-25 min) and the purified human fibrin/ogen was added to the warmed MOPS buffer and agitated at 37° C. until dissolved. One milliliter of PBS, pH 7.4 was added to plasmin to reconstitute, then 5 units of plasmin were added to fibrin/ogen in MOPS solution. This mixture was then agitated in a 37° C. incubator for 3 hr. The digested FDP were removed from the shaker and 200 μl protease inhibitor cocktail was added and the solution mixed thoroughly. The total protein concentration of the concentrated stock FDP solution was then measured, and the FDP breakdown product signature was verified on a 4-20% gradient SDS-PAGE under denaturing and reducing conditions (see FIG. 2). The resultant FDP solution was stored at −40° C.

The FDP immunogen was also used as in the immunoaffinity purification of the antibody and as an FDP calibrator and control in the FDP detection system.

### Example 2

### Production of Rabbit Anti-FDP Antibodies

Rabbits were immunized with FDP immunogen prepared according to Example 1. Each rabbit received injection of an emulsion consisting of 1 mg immunogen in 1.0 to 1.5 ml phosphate buffer saline and equal volume of Complete Freund's Adjuvant for the first immunization. Three weeks after the injection, each rabbit was bled and the serum was assayed for antibodies against FDP. One week after the bleeding, a booster was given to each rabbit by injection of an emulsion consisting of 1 mg immunogen in 1 to 1.5 ml phosphate buffer saline and equal volume of Incomplete Freund's Adjuvant. The rabbits were maintained on a schedule of boosters and bleeds until they were no longer viable.

Rabbit serums obtained at various time intervals after immunization were assayed for the concentration of antibodies against FDP by performing a standard titer assay using an 96-well plate system. In a typical serum titer assay, 10 μg of immunogen was immobilized in the wells of a 96-well microtiter plate; blocked with FBS; then reacted with a 1:1 dilution series of the various rabbit serums; washed; detected with a horseradish peroxidase (HRP) coupled goat anti-rabbit antibody (Sigma); washed; incubated with a 3,3′,5,5′-tetramethylbenzidine (TMB) solution; stop solution, and read at OD 450 nm. The titer was determined by graphing the OD 450 nm value versus the dilution factor; then determining the inflection point of the curve.

The rabbit anti-FDP was immunoaffinity purified as in Example 6.

### Example 3

### Production of Chicken Anti-FDP Antibodies

Chickens were immunized with FDP immunogen prepared according to Example 1. Each chicken received injections of an emulsion consisting of 1 mg immunogen in 1.0 to 1.5 ml phosphate buffer saline and equal volume of Complete Freund's Adjuvant for the first immunization (day 0). Each chicken was given additional immunizations on days 35, 56, 77, and 98. The chickens were bled (serum collection) on days 26, 40, 60, 81, and 100. Serum response testing was begun on day 45. The first harvest of IgY occurred on day 53. The first affinity purification began on day 56.

Chicken serums obtained at various time intervals after immunization were assayed for the concentration of antibodies against FDP by performing a standard titer assay using an 96-well plate system. A subset of the chicken anti-FDP IgY antibodies were conjugated to HRP prior to the assay. In a typical serum titer assay, 10 μg of immunogen was immobilized in the wells of a 96-well microtiter plate; blocked with FBS; then reacted with a 1:1 dilution series of the various rabbit serums; washed; incubated with a TMB solution; stop solution, and read at OD 450 nm. The titer was determined by graphing the OD 450 nm value versus the dilution factor; then determining the inflection point of the curve.

The chicken anti-FDP was immunoaffinity purified as in Example 6.

### Example 4

### Production of Goat Anti-FDP Antibodies

Goats were immunized with FDP immunogen prepared according to Example 1. Each goat received injections of an emulsion consisting of 1 mg immunogen in 1.0 to 1.5 ml phosphate buffer saline and equal volume of Complete Freund's Adjuvant for the first immunization. Three weeks after the injection, each goat was bled and the serum was assayed for antibodies against FDP. One week after the bleeding, a booster was given to each goat by injection of an emulsion consisting of 1 mg immunogen in 1 to 1.5 ml phosphate buffer saline and equal volume of Incomplete Freund's Adjuvant. The goats were maintained on a regime of boosters and bleeds, until the animal was no longer viable.

Goat serums obtained at various time intervals after immunization were assayed for the concentration of antibodies against FDP by performing a standard titer assay using an 96-well plate system. In a typical serum titer assay, 10 μg of immunogen was immobilized in the wells of a 96-well microtiter plate; blocked with FBS; then reacted with a 1:1 dilution series of the various goat serums; washed; detected with a HRP-coupled rabbit anti-goat antibody (Sigma); washed; incubated with a TMB solution; stop solution, and read at OD 450 nm. The titer was determined by graphing the OD 450 nm value versus the dilution factor; then determining the inflection point of the curve.

The goat anti-FDP was immunoaffinity purified as in Example 6.

### Example 5

### Preparation of FDP Coupled Sepharose CL 4B

Periodate-oxidized Sepharose CL 4B (20 ml) was washed with 10 times the Sepharose gel volume (i.e., ten times 20 ml of Sepharose gel which equals 200 ml) of Phosphate Buffered Saline (PBS) pH 7.5. The washed and drained Sepharose CL 4B was mixed with 20 ml (1 gel volume) of a 2.0 mg/ml solution of the pooled FDP antigens (FDP in MOPS, see Example 1). Three and a half milliliters (⅙^{th} of the gel volume) of 0.1M sodium cyanoborohydride in deionized (DI) water was added to this suspension and the suspension was shaken at room temperature for 16-20 hrs. Then 40 ml (2 times the gel volume) of 0.1M glycine and 0.1M sodium cyanoborohydride in PBS pH 7.5 was added to the suspension and the suspension was continuously agitated for an additional 2 hrs at room temperature. The FDP-coupled Sepharose CL-4B gel was degassed for 15 min and then packed in a chromatographic column by gravity flow. The gel was washed sequentially with the following solutions: DI water (10 times the gel volume), PBS pH 7.5 (10 times the gel volume), PBS containing 0.5M NaCl (10 times the gel volume), 0.1M sodium acetate/0.15M NaCl pH 3 (10 times the gel volume), PBS pH 7.5 (10 times the gel volume). When not in use, the gel was stored in PBS containing 0.1% sodium azide at 4° C. for up to one week.

### Example 6

### Immunoaffinity Purification of Polyclonal Antibodies Against FDP

Polyclonal antiserum was filtered through a 0.2 μm syringe filter prior loading it onto the FDP-coupled Sepharose CL 4B gel column prepared as in Example 5. The volume of filtered polyclonal antiserum that was added is approximately equal to the volume of the gel (1 gel volume). After the polyclonal antiserum has been added, the FDP-coupled Sepharose CL 4B gel column was washed again with PBS (5 times the gel volume) and then PBS containing 0.5M NaCl (5 times the gel volume). The immunoaffinity purified anti-FDP antibodies that bind to the FDP on the column were eluted with 0.1M sodium acetate/0.15M NaCl pH 3. The eluent was collected in the fraction tubes for monitoring and later those fractions with detectable protein concentrations (OD 280 nm greater than 0.2) were transferred to a PETG bottle for long term storage. The pH of the resultant antibody solution was neutralized by adding 1M Tris buffer pH 8.5.

### Example 7

### Characterization of Rabbit Polyclonal Antibodies Against FDP

In one embodiment, the capture antibody is an affinity purified, polyclonal rabbit antibody to FDP. The rabbits were immunized and the antibodies isolated as in Example 3.

SDS-PAGE of the affinity purified antibodies demonstrated a high degree of purity. The major protein band in the non-reduced anti-FDP preparation had a molecular weight of approximately 152 kDa, which is consistent with the molecular weight of rabbit IgG. Upon reduction, the protein band of 152 kDa disappeared and, simultaneously, two protein bands of approximately 59 kDa and of 30 kDa appeared, which are similar to the molecular weights of the heavy and the light chains of an IgG molecule, respectively. Therefore, the anti-FDP antibodies were primarily IgG, not IgM (M.W. approx. 900 kDa), nor IgA (a dimer of about 320 kDa). The purity of the affinity-purified FDP antibody pool was qualitatively estimated at about 90%, based on SDS-PAGE analysis.

The binding of anti-FDP antibodies to FDP antigens followed a typical Langmuir adsorption isotherm. From this data, using a double reciprocal plot, the binding constant of the antibodies was calculated to be 1.25×10⁻⁹ M. Because the antibody preparation was of polyclonal origin, the binding constant obtained in this study was a composite binding constant, i.e. an average of binding constant contributed by antibodies produced by different clones.

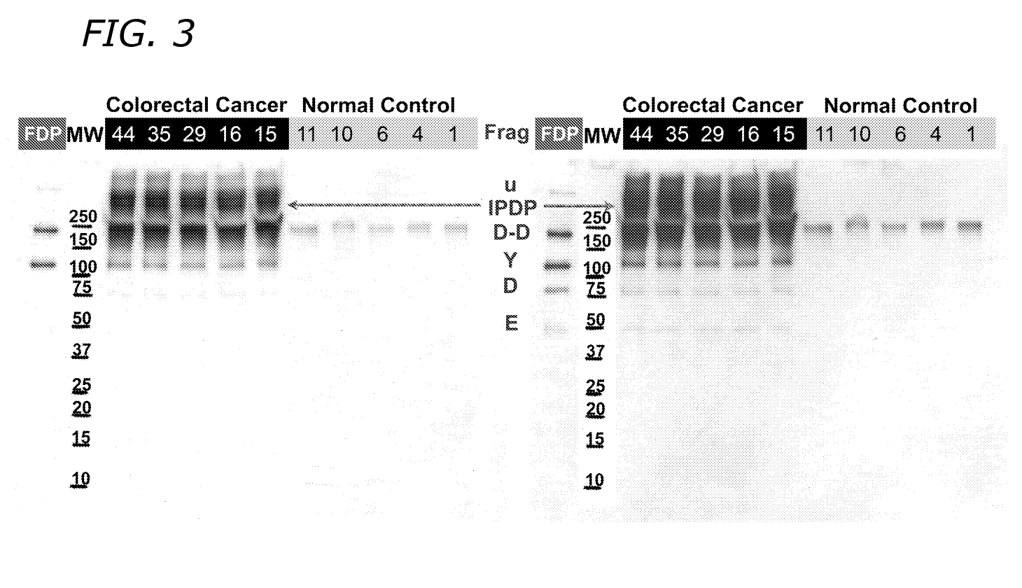

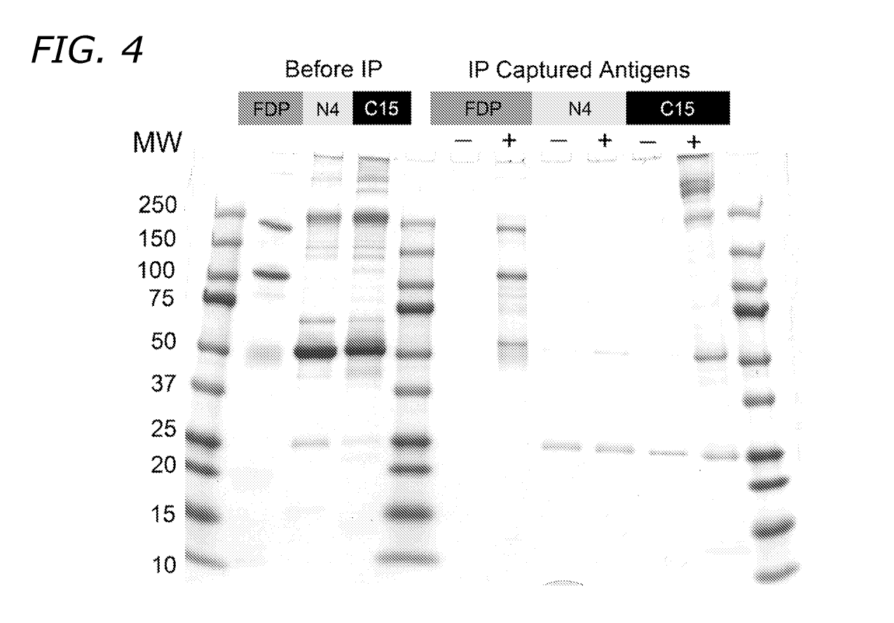

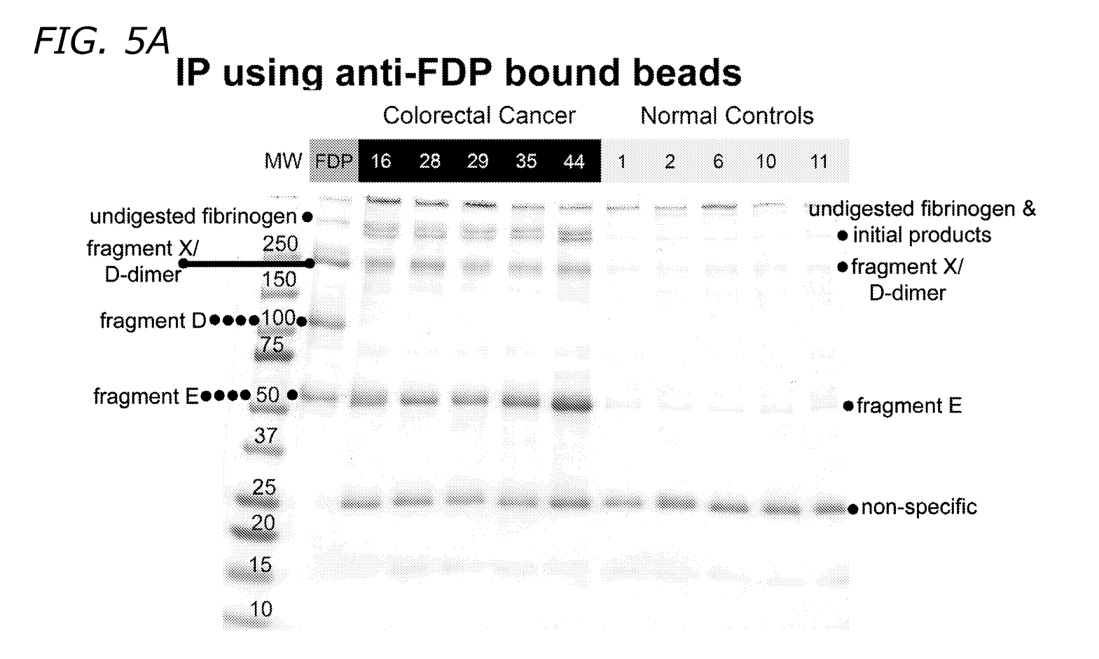

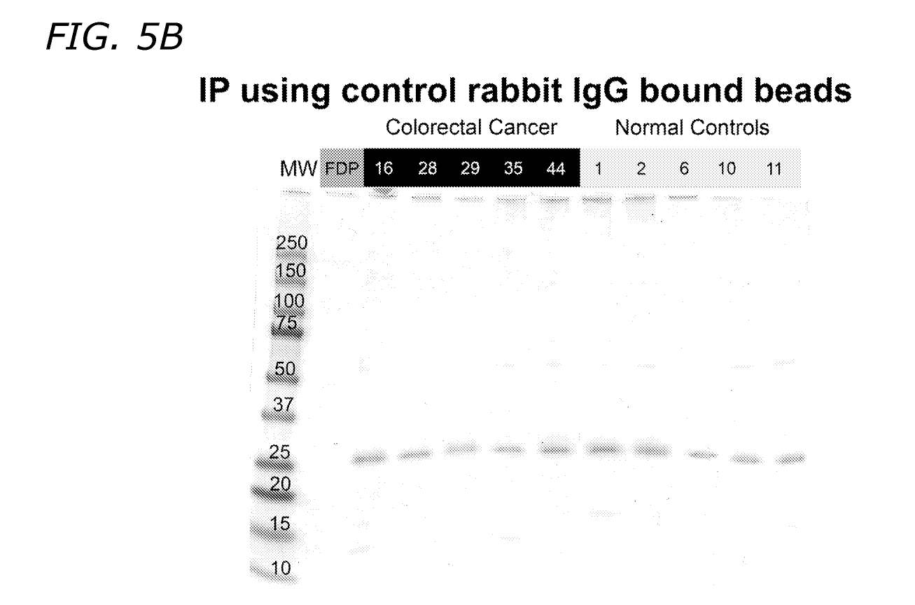

The capture and detection antibodies ensure the specificity of any ELISA based test. The FDP antibody pool was cross-reacted with human serum samples in both Western Blots (FIG. 3) and in immune precipitation (IP) experiments (FIGS. 4 and 5 and Table 2) in order to verify that the FDP antibody pool captures FDP in human serum samples. In addition, the proteins that were immunoprecipitated by the FDP antibody pool were sequenced and identified using mass spectrometry.

Western blot analysis of human serum samples from both colorectal cancer (CRC) and normal control patients were used to determine the number and the estimated molecular weights of the FDP antigens in human serum. FIGS. 3A and 3B depict Western blots of the same gel exposed to film for 5 sec and 30 sec respectively. FIG. 3C depicts the corresponding Coomassie-stained gel. In FIG. 3, six major FDP antigens were detected in CRC serum samples with estimated molecular weights of 340, 260-320, 220, 150, 80 and 50 kDa. These bands correspond with undigested fibrinogen, initial plasmin digest products (IPDP), fragment D-dimer/fragment X, fragment Y, fragment D and fragment E, respectively. In contrast, only one major band, at approximately 220 kDa, was detected in normal control sera. For all of the serum samples tested, the intensity of the reactions was much higher in the serum samples from CRC patients than from the normal controls.

TABLE 1
Analysis of anti-FDP cross reactivity in cancer vs. normal serum

| | | | Average of the Mean | | | |
|---|---|---|---|---|---|---|
| | | | Densities from | | | |
| Fibrinogen | Estimated | | Individual Subjects | | Ratio of Averaged | |
| Degradation | MW | Area | Cancer | Normal | Mean Densities | |
| Products | (kDa) | (in²) | n = 5 | n = 5 | Cancer/Normal | p-value |
| Undigested | 340 | 0.1 | 50.3 | 4.0 | 12.6 | 5.1E−08 |
| & IPDP | | | | | | |
| IPDP | 260-320 | 0.4 | 150.7 | 4.1 | 36.7 | 1.3E−10 |
| Fragment X/ | 220 | 0.2 | 131.1 | 14.8 | 8.8 | 4.0E−10 |
| Fragment D- | | | | | | |
| dimer | | | | | | |
| Fragment Y | 150 | 0.2 | 77.4 | 5.1 | 15.2 | 5.4E−10 |
| Fragment D | 80 | 0.2 | 51.0 | 3.8 | 13.5 | 5.4E−09 |
| monomer | | | | | | |
| Fragment E | 50 | 0.2 | 39.1 | 3.3 | 11.9 | 5.0E−08 |
| monomer | | | | | | |

As demonstrated in Table 1, the average signal densities of all six of these FDP components were significantly (p-values≦0.00000005) higher in the serum samples from the five CRC patients than from the five normal control patients. Densitometry-based analysis of cross-reactivity levels in the Western blot (FIG. 3A) used Scion Image densitometry software (Scion Corporation). Table 1 confirms that anti-FDP pool can distinguish between cancer and normal serum samples, based on their respective FDP levels.

In order to verify the number and sizes of the FDP antigens captured from human serum samples, immune precipitation studies were performed using either rabbit anti-FDP IgG or control rabbit IgG bound to sepaharose beads (FIGS. 4 and 5). Immune precipitation also concentrated the FDP antigens such that they could be sequenced from a Coomassie stained SDS-PAGE.

Using the Affi-Gel® Hz Immunoaffinity Kit (BIO-RAD) according to the manufacturer's instructions, the rabbit anti-FDP IgG or the control rabbit IgG antibodies were covalently linked to Sepharose beads via a conserved carbohydrate moiety in the Fc portion on the IgG heavy chain. Human serum samples were selected at random and diluted 1 to 10 in PBS, pH 7.4. The diluted serum samples were incubated with either the anti-FDP-bound beads or control rabbit IgG-bound beads. In addition, both types of beads were incubated with a 0.25 mg/ml solution of prepared FDP to serve as a positive control for the IP reactions. The IP reactions between the diluted sera and the control rabbit IgG bound beads serve as a control for recognizing false positive reactions between the non-specific rabbit IgGs and serum antigens. The IP reactions were incubated overnight at 4° C. on an end-over-end rotator. The IP beads were washed five times with PBS, and the antigens were extracted in non-reducing Laemmli sample buffer at 95° C. for 8 minutes. Then the extracted antigens from the anti-FDP and control rabbit IgG beads were run on a 4-20% SDS-PAGE. FIG. 4 shows a complete set of experimental conditions for two representative serum samples; N4 is from normal patient 4 and C15 is from colorectal cancer patient 15. FIGS. 5A and 5B reveal the reproducibility of these IP reactions by showing five additional normal and five additional colorectal cancer patients in the IP assay.

The results of the IP experiment, presented in FIG. 4, reveal that four major FDP-specific antigens and one non-specific antigen have been captured from the serum of the colorectal cancer patient serum by the anti-FDP beads (+). From this sample (C15(+), 2^{nd} to last lane from the right), the estimated molecular weights (MW) of the four major FDP-specific antigens are 340, 300, 220, and 50 kDa, respectively, in descending order. The one non-specific antigen in this lane is approximately 25 kDa. The control lanes N4(−) and C15(−) (3^{rd} and 5^{th} lanes from the right) for the serum samples also show a reaction with a 25 kDa protein, which corresponds with the 25 kDa protein in the N4 and C15 samples exposed to anti-FDP beads (+), as shown in N4(+) and C15(+) (2^{nd} and 4^{th} lanes from the right). The presence of the 25 kDa protein in all of these lanes, N4(−), N4(+), C15(−), C15(+), confirms that the 25 kDa protein extracted from the IP reactions of serum samples non-specifically interacts with any IgG bound bead. The positive control reactions for the IP assay are designated FDP(−) and FDP(+) (6^{th} and 7^{th} lanes from the left). The bead control lane (FDP(−)) reveals no interactions between the FDP calibrator solution, which is not serum based, and the negative control beads. The FDP(+) reaction contains one minor band and three major bands. The estimated MW of the one minor band is 340 kDa, which corresponds with the MW of undigested fibrinogen. The estimated molecular weights of the three major FDP-specific antigens in the FDP control immunogen solution are 220 kDa (which corresponds with the MW of the fibrinogen fragment X or D-dimer), 80 kDa (which corresponds with the MW of fibrinogen fragment D monomer) and 50 kDa (which corresponds with the fibrinogen fragment E monomer). The anti-FDP beads captured four major FDP antigens from the human serum of a colorectal cancer patient; whereas, the same beads only brought one minor (faint band) in the normal serum at approximately 53 kDa. Comparisons between the IP reactions of the human serum samples (and FDP) with the anti-FDP beads versus the control beads confirms the specificity of the anti-FDP antibody pool.

FIG. 5 reveals that the pattern of FDP antigens is reproducible among multiple patient samples. For this experiment, sera from an additional five CRC and five normal patients (chosen at random from clinical trial samples) were tested in immune precipitation reactions, separately, with both the control rabbit IgG and the anti-FDP beads. In addition, the FDP immunogen (calibrator solution) was tested with both types of beads. The SDS-PAGE in FIG. 5A contains the extracted FDP-specific antigens from the FDP (positive control), the CRC patient serum samples, and the normal serum samples. The SDS-PAGE shown in FIG. 5B shows the antigens adhering to the control rabbit IgG beads from the same samples and in the same order as in FIG. 5A.

The experiment shown in FIG. 5 confirms that there are four major FDP-specific antigens in human sera, and one non-specific antigen at 25 kDa. In FIG. 5A, the FDP control lane, which is derived from an in vitro plasmin digestion of fibrin and fibrinogen under controlled conditions, contains three major bands and one minor band. Based on the approximate molecular weights of these non-reduced samples, the major bands captured by the FDP antibody pool from the FDP control sample correspond to fibrinogen fragment D-dimer, fragment D monomer, and fragment E monomer, in descending order relative to the molecular weights. The minor band at 340 kDa is presumably undigested fibrinogen, also based on its approximate molecular weight. The FDP-specific antigens from human sera are at approximately 340, 300 (280-320), 210, and 50 kDa. These bands correspond with undigested fibrinogen, initial plasmin digest products (IPDP), fragment D-dimer/fragment X, and fragment E, respectively. The IP reactions in this (and all of the other IP experiments) were normalized by adding equivalent concentrations of each serum sample to the IP reactions. In FIG. 5B, the predominant antigen is at approximately 25 kDa. As discussed previously, this indicates that the serum protein at 25 kDa reacts non-specifically with all rabbit IgG bound beads. The 25 kDa protein also apparently interacts non-specifically with the rabbit anti-FDP IgG bound bead reactions shown in FIG. 5A.

TABLE 2
Quantification of DR-70 ® (FDP) - specific antigens from CRC patient sera

| | | | Average of the Mean | | | |
|---|---|---|---|---|---|---|
| | | | Densities from | | | |
| Fibrinogen | Estimated | | Individual Subjects | | Ratio of Averaged | |
| Degradation | MW | Area | Cancer | Normal | Mean Densities | |
| Products | (kDa) | (in²) | n = 5 | n = 5 | Cancer/Normal | p-value |
| Undigested | 340 | 0.1 | 42.5 | 7.1 | 6.0 | 1.5E−06 |
| fibrinogen | | | | | | |
| IPDP | 280-320 | 0.3 | 61.8 | 7.8 | 7.9 | 4.2E−08 |
| Fragment X | 210 | 0.3 | 57.0 | 8.7 | 6.6 | 3.9E−09 |
| & Fragment | | | | | | |
| D-dimer | | | | | | |
| Fragment E | 50 | 0.3 | 69.0 | 9.2 | 7.5 | 3.5E−05 |
| Non-specific | 25 | 0.3 | 64.1 | 68.1 | 0.9 | 5.3E−01 |

In this experiment shown in FIG. 5, the FDP specific antigens from the CRC patient sera were between 6.6 and 9.8 times more abundant than that from the normal patient sera, based on densitometry readings presented in Table 2. However, the non-specific antigen (approximately 25 kDa) had a ratio of cancer relative to non-cancer that was equal to 0.9 or approximately 1. As demonstrated in Table 2, the average signal densities of all of the FDP-specific antigens were significantly (p-values≦0.000035) higher in the serum samples from the five colorectal cancer patients than from the five normal control patients. The non-specific antigen band from the sera of cancer patients was not significantly (p=0.53) different from that of normal controls. Table 2 confirms that anti-FDP antibody pool can distinguish between cancer and normal serum samples based on their respective FDP levels.

### Example 8

### Characterization of the Anti-FDP Capture Antibody

The capture antibody pool was produced and characterized in one embodiment as described in Examples 2 and 7.

### Example 9

### FDP Antibody Conjugated to Horseradish Peroxidase (HRP) for Detection

The detection antibody was enzyme labeled using horseradish peroxidase (HRP) for detection in the assay. More specifically, in this example the detection antibody was a polyclonal rabbit anti-human FDP antibody produced according to the methods of Example 2. The antibody is the purified immunoglobulin fraction of rabbit antiserum conjugated with horseradish peroxidase of very high specific enzymatic activity.

The detection antibody immunogen included both fibrin and fibrinogen degradation products. The antibody reacts with native human fibrinogen as well as with the FDP fragment subtypes: D, E, X/D-dimer, and Y. Traces of contaminating antibodies were removed by solid-phase absorption with human plasma proteins. The specificity of the antibody was ascertained by Western blot analysis. The detection antibody has confirmed cross-reactivity with the same six FDP fragments in human serum of cancer patients as was detected by the capture antibody.

### Example 10

### Enzyme Linked Immunosorbant Assay (ELISA) for FDP

The FDP ELISA is an enzyme-labeled, sandwich immunoassay. The capture antibodies (polyclonal, rabbit anti-FDP) were derived from sera of rabbits as described in Examples 2, 7 and 8.

In an exemplary embodiment, the FDP immunoassay involves the use of removable strips in a 96-well, microtiter plate format. The wells of the microtiter plate were coated with affinity purified rabbit anti-FDP antibodies. Human serum samples were diluted (1:200) and were applied to the wells. FDP was captured from the serum samples by the anti-FDP antibodies immobilized on the well of the microtiter plate. After a wash step, anti-FDP antibodies conjugated to HRP (detection antibody) were added to the wells. The anti-FDP-HRP complex binds to the captured FDP to form an immunological sandwich with the immobilized anti-FDP antibodies. After a second wash step, the enzyme substrate TMB was added to the well. The end point was read in a micro plate reader at 450 nm after the reaction was stopped with 0.1N HCl. The intensity of the color formed is proportional to the amount of FDP in the serum. The amount was quantified by interpolation from a standard curve using purified FDP calibrators.

### Example 11

### Distribution of FDP in Serum from Normal Healthy Individuals and in Patients with Benign Disease

To determine the approximate distribution of FDP values in a normal healthy cohort, a sample of 209 women and 212 men who thought themselves to be disease free on the day of the draw was utilized. Subjects were divided by sex and by age with the two age groups: 40-64 years and 65 years plus. Values for the assay were determined in duplicate. A cumulative distribution was established. Order statistics for every 5th percentile were determined.

A variety of samples from subjects with benign disease were assembled to determine the distribution of serum FDP values in benign diseases that may be co-existent in patients with confirmed cancer. Specimens were collected under IRB approved protocols with the appropriate informed consent from geographically diversified sites across the United States. Table 3, below, describes the numbers of samples and the makeup of the benign disease groups. A total of 400 patients were tested in this cohort including 74 normal samples.

TABLE 3
Distribution of Diseases

| Benign Condition | Number |
|---|---|
| Benign Gastrointestinal (GI) Disease | 61 |
| Benign Genitourinary (GU) Disease | 94 |
| Heart Disease/HTN | 87 |
| Normal | 74 |
| Benign Pancreatic Disease | 84 |
| Grand Total | 400 |

Table 4 shows the characteristics of each of the benign groups tested as well as those for normal individuals. There is significant scatter within all the groups with the exception of the normal cohort. Mean values tend to be skewed by samples with high FDP values. However, the median value in each and every one of these groups is well within the reference interval for apparently healthy individuals. As in all other studies in this trial, FDP values were determined in duplicate with all subjects consented under an IRB approved protocol.

Analysis shows that the median values of these benign disease cohorts are of the same magnitude as the median value of the normal healthy subjects. All benign conditions except for those of the pancreas have distributions and 95% confidence limits similar to the normal healthy cohort.

TABLE 4
Sample Statistics by Benign Disease State

| Disease State | | FDP |
|---|---|---|
| Normal | N | 74 |
| | Mean | .81 |
| | Median | .77 |
| | Std. Deviation | .284 |
| | Minimum | .18 |
| | Maximum | 1.80 |
| Heart Disease | N | 87 |
| | Mean | 163.6 |
| | Median | 1.0 |
| | Std. Deviation | 337.53 |
| | Minimum | .14 |
| | Maximum | 1864.8 |
| Benign Pancreatic | N | 84 |
| Disease | | |
| | Mean | 262.8 |
| | Median | 1.2 |
| | Std. Deviation | 529.00 |
| | Minimum | .29 |
| | Maximum | 1902 |
| Benign GI Disease | N | 61 |
| | Mean | 29.3 |
| | Median | .798 |
| | Std. Deviation | 138.59 |
| | Minimum | .30 |
| | Maximum | 924.3 |
| Benign GU Disease | N | 94 |
| | Mean | 10.9 |
| | Median | .63 |
| | Std. Deviation | 98.89 |
| | Minimum | .17 |
| | Maximum | 959.57 |
| Total | N | 400 |
| | Mean | 97.9 |
| | Median | .81 |
| | Std. Deviation | 314.4 |
| | Minimum | .14 |
| | Maximum | 1901. |

### Example 12

### Distribution of FDP in Serum from Subjects with Malignant Disease

This study was conducted in 439 patients who had been diagnosed with, and were being treated for, a primary malignancy. The subjects were categorized into five groups: lung/liver cancer, breast/ovarian/cervical cancer, gall bladder/biliary/pancreatic cancer, gastric cancer and colorectal cancer. The FDP assay was performed in duplicate on the samples. A one-way ANOVA was performed with post hoc Tamhane tests. The results indicate that there are no significant differences between the groups. The details are shown in Table 5 for each cancer group.

TABLE 5
Sample Statistics by Cancer Group

| Cancer Group | | FDP |
|---|---|---|
| Lung/Liver Cancer | N | 89 |
| | Mean | 4.05 |
| | Median | 1.65 |
| | Std. Error of Mean | 1.10 |
| | Minimum | .17 |
| | Maximum | 76.12 |
| Breast/Cervical/Ovarian | N | 90 |
| Cancer | Mean | 3.43 |
| | Median | 1.67 |
| | Std. Error of Mean | .53 |
| | Minimum | .31 |
| | Maximum | 39.23 |
| Gallbladder/Pancreas Cancer | N | 47 |
| | Mean | 3.54 |
| | Median | 2.29 |
| | Std. Error of Mean | .75 |
| | Minimum | .64 |
| | Maximum | 34.00 |
| Gastric/Other Cancers | N | 26 |
| | Mean | 34.99 |
| | Median | 2.24 |
| | Std. Error of Mean | 32.22 |
| | Minimum | .67 |
| | Maximum | 840.54 |
| Colorectal Cancer | N | 187 |
| | Mean | 3.43 |
| | Median | 1.33 |
| | Std. Error of Mean | 1.38 |
| | Minimum | .24 |
| | Maximum | 259.28 |
| Total Cancers | N | 439 |
| | Mean | 5.44 |
| | Median | 1.61 |
| | Std. Error of Mean | 2.01 |
| | Minimum | .17 |
| | Maximum | 840.54 |

### Example 13

### Distribution in Percent of FDP Values within Each Disease Cohort

In each normal, benign and disease cohort above, the values for FDP were analyzed for the different FDP concentration levels within each disease cohort. The StatXact® software was utilized during this analysis to establish the exact 95% confidence intervals for the statistics. The distribution table is presented in Table 6.

TABLE 6
Distribution of percent of FDP values

| | | Percent (%) | | | |
|---|---|---|---|---|---|
| | # of | 95% CI (lower %, upper %)* | | | |
| Disease | subjects | 0-1.4 μg/ml | 1.5-2.4 μg/ml | 2.5-4.9 μg/ml | ≧5.0 μg/ml |
| Normal | 420 | 94.5 | 5.0 | 0.5 | 0.0 |
| | | (91.9, 96.5) | (3.1, 7.5) | (0.1, 1.7) | (0.0, 0.9) |
| <65 years | 337 | 96.4 | 3.3 | 0.3 | 0.0 |
| | | (93.9, 98.2) | (1.6, 5.8) | (0.0, 1.6) | (0.0, 1.1) |
| ≧65 years | 83 | 86.8 | 12.1 | 1.2 | 0.0 |
| | | (77.5, 93.2) | (5.9, 21.0) | (0.0, 6.5) | (0.0, 4.4) |
| Benign | 326 | 75.5 | 6.8 | 0.6 | 17.2 |
| | | (70.4, 80.0) | (4.3, 10.0) | (0.1, 2.2) | (13.2, 21.7) |
| GU Disease | 94 | 94.7 | 4.3 | 0.0 | 1.1 |
| | | (88.0, 98.3) | (1.2, 10.5) | (0.0, 3.9) | (0.0, 5.8) |
| GI Disease | 61 | 90.2 | 3.3 | 0.0 | 6.6 |
| | | (79.8, 96.3) | (0.4, 11.4) | (0.0, 5.9) | (1.8, 16.0) |
| Pancreas | 84 | 60.7 | 15.5 | 2.4 | 21.4 |
| | | (49.5, 71.2) | (8.5, 25.0) | (0.3, 8.3) | (13.2, 31.7) |
| Heart Disease | 87 | 58.6 | 3.5 | 0.0 | 37.9 |
| | | (47.6, 69.1) | (0.7, 9.8) | (0.0, 4.2) | (27.7, 49.0) |
| Malignant | 439 | 44.0 | 24.2 | 19.6 | 12.3 |
| | | (39.3, 48.8) | (20.2, 28.4) | (16.0, 23.6) | (9.4, 15.7) |
| Colon | 187 | 55.6 | 21.4 | 15.0 | 8.0 |
| | | (48.2, 62.9) | (15.7, 28.0) | (10.2, 20.9) | (4.6, 12.9) |
| Lung | 44 | 34.1 | 38.6 | 18.2 | 9.1 |
| | | (20.5, 49.9) | (24.4, 54.5) | (8.2, 32.7) | (2.5, 21.7) |
| Liver | 44 | 31.8 | 27.3 | 22.7 | 18.2 |
| | | (18.6, 47.6) | (15.0, 42.8) | (11.5, 37.8) | (8.2, 32.7) |
| Breast | 31 | 54.8 | 25.8 | 12.9 | 6.5 |
| | | (36.0, 72.7) | (11.9, 44.6) | (3.6, 29.8) | (0.8, 21.4) |
| Ovarian | 31 | 25.8 | 6.5 | 32.3 | 35.5 |
| | | (11.9, 44.6) | (0.8, 21.4) | (16.7, 51.4) | (19.2, 54.6) |
| Cervical | 28 | 50.0 | 28.6 | 7.1 | 14.3 |
| | | (30.7, 69.4) | (13.2, 48.7) | (0.9, 23.5) | (4.0, 32.7) |
| Gall Bladder | 19 | 42.1 | 26.3 | 31.6 | 0.0 |
| | | (20.3, 66.5) | (9.2, 51.2) | (12.6, 56.6) | (0.0, 17.7) |
| Pancreas | 28 | 25.0 | 17.9 | 35.7 | 21.4 |
| | | (10.7, 44.9) | (6.1, 36.9) | (18.6, 55.9) | (8.3, 41.0) |
| Gastric/Other | 27 | 22.2 | 33.3 | 29.6 | 14.8 |
| | | (8.6, 42.3) | (16.5, 54.0) | (13.8, 50.2) | (4.2, 33.7) |

*Exact binomial confidence limits.

### Example 14

### Serial (Longitudinal) Monitoring of Patients Diagnosed with Colorectal Cancer

Samples for this portion of the study were obtained from two retrospective sample banks. Forty-eight serial sets were obtained from Geffen Cancer Center in Vero Beach, Fla. and sixty-four serial sets were taken from the serum banks at MD Anderson Cancer Center in Houston, Tex.

Out of a total of 445 evaluable observations, there were 112 evaluable patient serial sets. The samples for the serial monitoring study were retrospective banked samples that were collected blindly and without bias to include all patients with diagnosed colorectal cancer in the bank at the time of the collection. The average number of observations per patient is 4.0.

In summary, serial samples were taken from 112 colon cancer patients resulting in a total of 445 paired observations in which a FDP reading and a determination of disease progression were obtained. The sequential draws covered an average longitudinal period of at least nine months. Progression of the FDP value in the serial monitoring set was evaluated as a percentage change between the current and previous readings (Y) and 15% was determined to be the minimum percentage to specify disease progression in the FDP assay. Clinical disease progression (D) was determined by the subject's physician based on those office procedures and clinical laboratory based analyses that were the standard of care during the time of the monitoring period. The primary objective of this analysis is to demonstrate that the FDP assay was informative by showing that the sum of sensitivity and specificity exceeds 1.

The response to therapy was evaluated using information provided in the records by the clinicians based on the results of clinical examinations and imaging results (i.e. bone scans, CT scans, magnetic resonance imaging studies, radiography, or ultrasound). Response to therapy is defined as follows:

Complete response or no evidence of disease (CR or NED): The complete disappearance of all clinical and image-measurable disease as evidenced by the clinical exam and imaging or other diagnostic modalities as ordered by the physician.

Partial Response (PR): In patients with metastases at the time of the original draw, a noticeable reduction in the size of primary metastatic lesions or bone metastases demonstrating at least stabilization as observed on the bone scan.

Stable Disease (SD): No significant change in the size of primary metastatic lesions or no noticeable increase in the size of primary lesions or no new lesions as evidenced by the clinical exam and imaging or other diagnostic modalities as ordered by the physician.

Progressive Disease (PD): Clinical or imaging results that clearly indicate the presence of lesions not seen on previous examinations or a significant increase in the size of primary or metastatic lesions.

Serial samples were taken from 112 colon cancer patients resulting in 446 paired observations in which the FDP assay reading and a determination of disease progression were obtained. Since several patients had signs of progression even at the first examination, it was decided to determine the relationship between FDP assay value and progression at successive visits. Thus, from the data, a variable was derived by taking the ratio of a subsequent FDP assay reading and the previous reading. This measure was intended to determine the increase from a previous reading as a means of providing information on progression. A determination was made that a meaningful increase to determine evidence of progression was 15% increase or more. Thus, if the ratio was 1.15 or higher, the FDP assay value was deemed to be positive, otherwise it was deemed to be negative and this determination was paired with the finding at that visit of progression or not.

The resulting 335 paired observations from the post baseline sampling were evaluated in two ways, per-visit and per-patient evaluations. The initial analysis performed a bootstrap sample for each patient by randomly sampling one visit at for each sample and recording the sensitivity or specificity for that visit. Note that if there was a progression and the sensitivity would be 1 if the FDP assay value increased from the previous visit by 15% or more and 0 if it did not. If there were no progression at that visit, then there would be no sensitivity reported at that visit, but the specificity would be reported as a 1 if the FDP assay value was below a 15% increase for that visit and 0 otherwise. For the per-visit analysis, there were 135 visits for sensitivity and 198 visits for specificity.

A second analysis was done on a per-patient basis in which the number of progressions across all visits for a given patient was used to compute a patient level sensitivity by taking the number visits that FDP assay values increase by at least 15% among the number of visits that there was a progression. Similarly, the number of visits at which FDP assay values had a lower than 15% increase divided by the number of visits in which there was a non-progression allowed the computation of a per-patient specificity. Recall that if a patient had all progressions there would be no specificity for that patient and if a patient had all non-progressions, there would be no sensitivity for that patient. This resulted in a sample of 112 patients with at least one sensitivity, specificity, or both. This resulted in 70 estimates of per-patient sensitivity and 86 estimates of per-patient specificity.

Positive (PPV) and negative (PNV) predictive values were also computed on a per-patient and per-visit basis. The PPV is calculated by dividing the number of times progression was present when the FDP assay value increased by 15% or more and NPV by dividing the number of times progression was absent when FDP assay values did not increase by 15% or more.

When small samples are available for evaluation and may have a complex structure, a statistical method called the bootstrap may be used to provide sound estimates of relevant population properties. A method to allow such a computation is to take repeated random samples with replacement from the 333 observations and compute the sum of the sensitivity and specificity and then determine through the frequency distribution of the resulting samples the sum that excludes the percentile corresponding to the alpha level of the statistical test. Such samples automatically incorporate any intra-patient correlations and yield an unbiased test of the null hypothesis above. Bootstrap sampling is usually repeated a number of times to be certain that the resulting distributions are consistent from run to run.

The per-visit bootstrap sample was done 2,000 times by randomly sampling one visit for each of the 112 patients with replacement. From each sample, the mean sensitivity, means specificity, and the sum were computed by summing the sensitivity or specificity (the 1 or 0 values) over all patients and dividing by the total number of sensitivity or specificity measurements present. After the mean sensitivity and specificity for a sample was obtained, the two were added together to obtain the sum. This exercise resulted in 2000 bootstrap estimates of the per-visit sensitivity, specificity, and the sum of the two. A frequency display of these 2000 values by variable allows the boot strap estimation of the lower one- and two-sided 95% confidence limits as well as other.

The method of sampling was done by assigning each of the k (between 1 and 7) visits per-patient a number between 1 and k. A sample of 112 observations was obtained by generating a uniform random number between 0 and 1, multiplying that number by k, taking the integer of that number, and adding 1. This process provided a random number between 1 and k for each patient. The FDP assay ratio of change from previous was captured for the patient visit with the number obtained. Duplicates were allowed because sampling was done with replacement and the process was repeated 112 times. The sensitivity, specificity, and the sum were computed for each sample of 112 and retained.

The computed per-visit sensitivity from the 333 per-visit evaluations was 100*88/134=65.19, the specificity was 100*133/199=67.34, the sum of sensitivity and specificity was 132.53, the PPV was 100*88/153=57.52, and the NPV was 100*134/181=74.03.

The results of the statistical analysis to determine the effectiveness of the FDP immunoassay for providing information on disease progression in colon cancer patients are provided in Table 7 below.

TABLE 7
Results of Five Repetitions of 2,000 Samples of 112 per-visit
Observations of the Sensitivity, Specificity, the Sum of
Sensitivity and Specificity, PPV, and NPV

| Run | Measure | Median | Lower 5% | Lower 2.5% |
|---|---|---|---|---|
| 1 | Sensitivity | 65.85 | 55.81 | 54.17 |
| | Specificity | 66.20 | 59.09 | 57.63 |
| | Sensitivity + Specificity | 132.66 | 120.31 | 118.23 |
| | PPV | 58.33 | 50.91 | 49.23 |
| | NPV | 73.13 | 66.18 | 65.45 |
| 2 | Sensitivity | 65.85 | 56.25 | 54.17 |
| | Specificity | 67.19 | 58.46 | 56.92 |
| | Sensitivity + Specificity | 132.92 | 120.75 | 118.72 |
| | PPV | 58.82 | 50.91 | 48.98 |
| | NPV | 73.21 | 66.18 | 64.91 |
| 3 | Sensitivity | 65.91 | 56.10 | 54.00 |
| | Specificity | 67.16 | 58.73 | 56.92 |
| | Sensitivity + Specificity | 133.20 | 120.29 | 117.81 |
| | PPV | 58.93 | 49.15 | 48.94 |
| | NPV | 73.33 | 66.18 | 65.08 |
| 4 | Sensitivity | 65.85 | 56.25 | 53.66 |
| | Specificity | 66.67 | 58.57 | 56.72 |
| | Sensitivity + Specificity | 132.96 | 119.73 | 116.99 |
| | PPV | 58.73 | 49.18 | 48.84 |
| | NPV | 73.13 | 66.00 | 64.52 |
| 5 | Sensitivity | 65.96 | 56.25 | 54.90 |
| | Specificity | 67.16 | 58.73 | 57.38 |
| | Sensitivity + Specificity | 133.29 | 120.62 | 118.23 |
| | PPV | 59.02 | 50.88 | 49.09 |
| | NPV | 73.24 | 66.20 | 64.91 |

Whether the test is done at the alpha=0.05 or at the alpha=0.025, the sum of sensitivity and specificity from this analysis clearly is statistically significantly above 100. The median sum is likely to be about 133 with the median sensitivity about 65 and the median specificity about 67. The lower one-sided 5% confidence bound for the sum is about 120 and the lower one-sided 2.5% confidence bound is about 118. The median PPV and NPV are about 59 and 73, respectively, across the five samples. Note that these values are consistent with those computed from the per-visit values given above in Table 7. The five repetitions of the sample of 2,000 demonstrate that the result is robust and consistent.

For the per-patient analysis, the computed per-visit sensitivity from the 112 per-patient evaluations was 100*45.68/69=66.21, the specificity was 100*58.63/86=68.18, the sum of sensitivity and specificity was 134.39, the PPV was 100*51.83/97=53.44, and the NPV was 100*71.67/103=69.58.

TABLE 8

| Run | Measure | Median | Lower 5% | Lower 2.5% |
|---|---|---|---|---|
| 1 | Sensitivity | 66.12 | 58.78 | 57.11 |
| | Specificity | 68.20 | 62.58 | 61.46 |
| | Sensitivity + Specificity | 134.38 | 123.82 | 121.46 |
| | PPV | 53.30 | 45.88 | 44.55 |
| | NPV | 69.74 | 62.71 | 60.90 |
| 2 | Sensitivity | 66.17 | 58.58 | 57.13 |
| | Specificity | 68.16 | 62.39 | 61.41 |
| | Sensitivity + Specificity | 134.26 | 123.85 | 122.17 |
| | PPV | 53.41 | 45.34 | 43.96 |
| | NPV | 69.50 | 62.75 | 61.37 |
| 3 | Sensitivity | 66.13 | 58.59 | 57.27 |
| | Specificity | 68.20 | 62.29 | 61.23 |
| | Sensitivity + Specificity | 134.32 | 123.79 | 121.36 |
| | PPV | 53.37 | 45.71 | 43.89 |
| | NPV | 69.50 | 62.06 | 60.89 |
| 4 | Sensitivity | 66.43 | 58.53 | 56.99 |
| | Specificity | 68.28 | 62.64 | 61.55 |
| | Sensitivity + Specificity | 134.69 | 123.87 | 121.17 |
| | PPV | 53.70 | 45.92 | 44.39 |
| | NPV | 69.55 | 62.71 | 61.33 |
| 5 | Sensitivity | 66.42 | 59.00 | 57.92 |
| | Specificity | 68.29 | 62.82 | 61.64 |
| | Sensitivity + Specificity | 134.62 | 124.38 | 122.92 |
| | PPV | 53.41 | 45.75 | 44.15 |
| | NPV | 69.64 | 62.66 | 61.38 |

Whether the test is done at the alpha=0.05 or at the alpha=0.025, the sum of sensitivity and specificity from this analysis clearly is statistically significantly above 100. The median sum is likely to be about 134 with the median sensitivity about 66 and the median specificity about 68. The lower one-sided 5% confidence bound for the sum is about 124 and the lower one-sided 2.5% confidence bound is about 121. The median PPV and NPV are about 53 and 69, respectively, across the five samples. Note that these values are consistent with those computed from the per-patient values given above Table 8. The five repetitions of the sample of 2,000 demonstrate that the result is robust and consistent.

The conclusion from the analysis of the effectiveness of the FDP immunoassay for monitoring colorectal cancer samples is as follows. These data and analyses demonstrate that the FDP assay, when taken as a 15% or greater change from the previous visit, yields informative data regarding colon cancer progression. The FDP immunoassay results must be used in conjunction with standard of care procedures for monitoring colorectal cancer patients.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method for detecting cancer in a subject, said method comprising the steps of:
obtaining a biological sample from said subject;
reacting said biological sample with an antibody preparation which binds at least three antigens associated with fibrin and fibrinogen degradation products (FDP) to form antibody-FDP complexes wherein said three FDP-associated antigens are fragment D, fragment E and D-dimer;
detecting said antibody-FDP complexes; and
diagnosing cancer in said subject.

2. The method according to claim 1 wherein said antibody preparation additionally optionally binds to at least one of fragment Y and initial plasmin digest products (IPDP).

3. The method according to claim 1 wherein said antibody preparation is a polyclonal antibody preparation.

4. The method of claim 1 wherein said method comprises an enzyme-linked immunosorbent assay.

5. The method of claim 1 wherein diagnosing step further comprises at least one additional diagnostic test.

6. A method of monitoring cancer in a patient comprising:
(a) obtaining a first biological sample from said subject, said first sample collected at a first sampling time point;
(b) obtaining a second biological sample from said subject, said second sample collected after said first sampling time point at a second sampling time point;
(c) reacting said biological samples with an antibody preparation which binds at least three antigens associated with FDP to form antibody-FDP complexes wherein said three FDP-associated antigens are fragment D, fragment E and D-dimer;
(d) detecting said antibody-FDP complexes; and
(e) determining the ratio of the level of FDP in said second biological sample to the level of FDP in said first biological sample;
(f) determining that cancer has progressed; and
optionally repeating steps (a)-(e) with additional biological samples taken at time points after said first and said second time points.

7. The method according to claim 6 wherein said antibody preparation optionally additionally binds to at least one of fragment Y and initial plasmin digest products (IPDP).

8. The method according to claim 6 wherein said antibody preparation is a polyclonal antibody preparation.

9. The method according to claim 6 wherein said reacting and detecting steps comprise an enzyme-linked immunosorbent assay.

10. The method according to claim 6 wherein said cancer has progressed if said ratio is greater than or equal to 1.15.

11. The method according to claim 6 wherein said cancer has regressed or is stable if said ratio is less than 1.15.

12. A kit for detecting cancer in a subject, said kit comprising:
an antibody preparation which binds to at least three antigens associated with FDP wherein said three FDP-associated antigens are fragment D, fragment E and D-dimer;
a detection system; and
instructions for measuring said FDP and correlating the presence of said FDP with cancer.

13. The kit according to claim 12 wherein said detection system comprises a detection antibody specific for at least three antigens associated with FDP wherein said three FDP-associated antigens are fragment D, fragment E and D-dimer.

14. The kit according to claim 13 wherein said antibody preparation optionally additionally binds to at least one of fragment Y and initial plasmin digest products (IPDP).

15. The kit according to claim 12 wherein said antibody and said detection system comprise an enzyme-linked immunosorbent assay.
